(19) United States
(12) Patent Application Publication
(10) Pub. No.: US 2014/0155914 A1
(43) Pub. Date: Jun. 5, 2014

(54) DEVICES AND METHODS FOR PASSING SUTURES THROUGH TISSUE

(71) Applicant: David James Schneider, Lafayette, CO (US)

(72) Inventor: David James Schneider, Lafayette, CO (US)

(21) Appl. No.: 13/958,401

(22) Filed: Aug. 2, 2013

Related U.S. Application Data
(60) Provisional application No. 61/679,368, filed on Aug. 3, 2012.

(51) Int. Cl. A61B 17/04; A61B 17/06

(52) U.S. Cl. CPC A61B 17/0483; A61B 17/06066; A61B 17/0401; A61B 17/06166
USPC 606/144; 606/228; 606/223

(57) ABSTRACT

A suture passer device is provided including a jaw configuration that grasps targeted tissue such as tendons or muscles in a similar manner as existing suture passer devices; however, the device introduces the needle and the attached suture along the axis of the shaft of the device. After grasping the targeted tissue, the needle is deployed by manipulating the handle of the device similar to conventional suture passers however, the needle is deployed in between the opposing jaws and the needle is advanced into tissue along the axis of the shaft as opposed to perpendicular to the shaft as in prior art devices. The invention further includes methods of deploying sutures and anchors. The invention also includes embodiments of jaw designs, and various embodiments of suture passing needles, sutures constructions, and anchor designs. Therefore, the invention also comprises the device in combination with the needles, sutures, and anchors, as well as the needles, sutures, and anchors in separate sub combinations.

### CROSS REFERENCE TO RELATED APPLICATION

This application is a non-provisional application that claims priority to provisional Application Ser. No. 61/679,368, filed on Aug. 3, 2012, entitled “Apparatus and Method for Passing Sutures Through Tissue”, this provisional application being incorporated by reference herein in its entirety.

### FIELD OF THE INVENTION

This invention relates to devices for passing of suture or like material in soft tissue, and the subsequent ability to repair or reattach the targeted tissue. The invention further relates to methods that employ use of the apparatus, and various components in sub-combination for use in the devices.

### BACKGROUND OF THE INVENTION

In rotator cuff repairs, suture is passed through the rotator cuff in order to secure the rotator cuff tendon back to the bone from which it was separated or torn. The action of “passing” suture must be utilized to apply tension and repair the rotator cuff. Most commonly, the suture that is passed through the rotator cuff is connected to a shoulder anchor that is inserted into the bone. After the suture is passed, the rotator cuff anchor deployed into the bone may require the surgeon to tie knots or utilize a knotless technique for securing the suture. The surgeon may also use bone tunnels instead of a shoulder anchor to secure the passed suture and tissue to the bone. In any of the current surgical techniques, the suture is used to tension and manipulate the rotator cuff tendon in an effort to secure it back to the bone. As arthroscopic surgery has become more popular for repair of rotator cuff injuries, passing suture through the rotator cuff has resulted in develop of instruments that allow the surgeon to pass suture through the rotator cuff more easily and efficiently. Often times, passing suture is a difficult step for a surgeon because the surgeon is limited by cannula placement, size of the portals, or angle of entry to access the rotator cuff. Additionally, the suture is optimally passed through the healthiest portion of the rotator cuff tissue, which is typically the further medial location on the rotator cuff to thereby increase the strength of the repair; however this portion of the tissue may be difficult to access considering limitations in the design and orientation of current tissue passing devices.

One common type of device used for passing suture through a rotator cuff is characterized by a device having a handle and a manually operated jaw structure located at the end of a shaft that allows the surgeon to pass suture into or near the desired area of the rotator cuff. Most current devices use a jaw construct to grasp the tissue with the use of a standard handle (ratcheting or non-ratcheting) that allows for a needle to pass from the distal ends of the jaw. The handle allows the surgeon to manipulate the jaws, grasp the tissue and advance the needle. The distal ends of the jaws are utilized to pass the needle after grasping the tissue because it typically accesses the healthiest portion of the patient's rotator cuff. Further, many current devices attempt to maximize the length of the jaw to pass the suture in a location on the rotator cuff as medial as possible. In use, the needle captures the desired suture (which is placed in the jaw prior to passing) as the needle advances from the bottom of the jaw to the top jaw. The device passes the suture through a single point in the rotator cuff that is limited by the length of the grasping jaws.

In most procedures, the needle is preferably passed from the bottom to the top jaw allowing the suture to be in view of the arthroscope after being passed, as opposed to passing from the top to bottom which will obstruct viewing of the suture. The suture is then retrieved with the jaws of the grasper device, and the rotator cuff is ready to be tensioned and the repair completed.

Other types of instruments for suture passing may allow the passage of the needle and the retrieval of the suture in one step, or may allow for simultaneous passages of multiple sutures. Current devices in use may have the following features or characteristics: (1) The needle and suture are passed nearly perpendicular to the plane of the grasping jaw, the term “nearly” being used because most suture needles have a bend located at a point where the needle extends from a corner from the bottom jaw to the top jaw; (2) Commonly, the needle travels along the long axis of the jaw, but deforms or curves near the distal tip of the instrument to penetrate tissue (most commonly from bottom to top or inferior to superior); (3) The suture is typically routed from inferior to superior or vice versa after being passed, or from the underside of the rotator cuff to the top side of the rotator cuff; (4) The suture may be a braided construct and may vary in thickness; (5) The suture can easily slide back and forth through the rotator cuff after being passed; and (6) The suture may be passed perpendicular or transverse to the fibers of the rotator cuff.

The current method of passing suture results in a simple, single point of fixation between the suture and rotator cuff. Because of this single point of fixation, the suture can commonly pull through the targeted tissue under tension and therefore the repair can be compromised. The use of high strength sutures can further increase the incidence of the suture pulling through or “cutting through” the tissue. This incidence results in the suture remaining intact while the rotator cuff is compromised by the cutting action of the suture. This type of failure in a rotator cuff surgery is described as suture pulling through the rotator cuff without the implant or the suture failing. Hence, the patient is left with a rotator cuff repair that has failed yet the suture and anchor remain intact. Many surgeons also desire to maximize the contact surface from the single point of suture passage to the anchor or bone tunnel. Because the single point of suture fixation results in suture on the underside (inferior) of the rotator cuff, this can result in reduced contact surface area between the rotator cuff and bony surface. The contact surface area is reduced because suture remains present between the rotator cuff and bony reattachment site from the point the suture is passed to the position where the implant is placed. In addition, the shape of the needle passed in current devices, is sharp and flat in shape, not curved or rounded which may increase the trauma placed on the tissue. Because of current needle geometry, it typically cuts against the alignment of the rotator cuff fibers. The cutting aspect of the needle can increase the likelihood of creating a point of failure because of the manner in which it enters and passes through the rotator cuff. Current techniques also place the suture between tissue and bone reducing the contact surface and possible healing sites.

Some current devices allow for two needles to be passed in the same manner as described above. There are other devices available that pass suture from one side of tissue to the other (inferior to superior or vice versa passage) with the use of a penetrating type device and a mechanism to grasp the suture. The penetrating type device includes a sharp point that penetrates the tissue, and another part of the device is positioned to then retrieve or pass the desired suture.

There are also multiple suture constructs that are made of different materials, sizes, and configurations. Most sutures have a braided construction that may vary in thickness to accommodate surgeon preferences. In some cases, sutures have barbed type configurations to better secure tissue and reduce the migration of sutures after being advanced in the tissue. The barbed type of suture is typically used for dermal or fascia type applications, but not utilized in soft tissue reattachment to bone. The current barbed suture constructs are attached to standard cutting needles and are passed in a traditional open surgical method. This type of suture is not known to be utilized in conjunction with a suture passing device and specifically with a suture passing device having the above described jaw configuration.

Although the passing of suture has been simplified with recent devices, the current devices still rely on a single point of passage through the rotator cuff. The point in which suture is passed results in a single point of fixation, and a significant portion of the subsequent strength is determined by the interface between the suture and soft tissue at that single point. The geometry of current needles also have a tendency to cut through a tendon at the point of passage in a flat cutting direction opposite of the direction in which tendon fibers extend. In some cases, rotator cuff repairs will fail as mentioned because suture will ‘cut’ through the rotator cuff after being violated by this type of needle. The devices that utilize a jaw construct are also limited by the size or length of the jaws. Therefore, the depth in which the surgeon can access healthier or thicker portions of the soft tissue to increase strength is limited. Many times with current devices, the surgeon will attempt to grasp as much tissue as the jaw construct will allow in an attempt to be as medial as possible and access potentially healthier tissue.

Understanding the nature of currently available devices and methods, it is apparent that there are still unmet needs regarding improvements in suture passing constructions and methods to overcome existing deficiencies associated with the prior art.

### SUMMARY OF THE INVENTION

In accordance with the present invention, a suture passer device is provided including a jaw configuration that grasps the rotator cuff in a similar manner as existing suture passer devices; however, the device introduces the needle and the attached suture along the axis of the shaft between the opposing jaws. More specifically, after grasping the rotator cuff, the needle is deployed by manipulating the handle of the device similar to conventional suture passers however, the needle is deployed in between the opposing jaws and the needle is advanced into tissue along the axis of the shaft as opposed to perpendicular to the shaft as in prior art devices. Introducing the needle along the axis and in between the opposing jaws in this manner allows a surgeon to advance a suture through the central portion of the rotator cuff. This orientation of the needle and suture along the length of the shaft also enables the surgeon to potentially advance the suture further into the medial aspect of the rotator cuff and to secure more soft tissue. The introduction of the suture into the central portion of the tissue also gives the surgeon more suture to tissue contact, allowing for more potential strength of the repair when securing the suture to a conventional implant. The present invention may also reduce the incidence of “cut through” of suture through the rotator cuff because the force and tension of the suture is not through a single point, but rather passed through the length of the tissue and tensioned along the axis of the tissue. In the same way, a needle does not lacerate the tendon in a smaller cross-sectional area of tissue like current devices. The orientation of the suture entry and advancement of the suture ultimately allows for suture fixation along the entire aspect of the repair site of a rotator cuff that provides an increase in strength of the repair because of the increased surface area between the suture and tissue.

Maximizing the contact surface area between the rotator cuff and the bone is also important in the success of rotator cuff repairs. The present invention reduces or eliminates the amount of suture located along the inferior side of the rotator cuff to thereby increase the contact surface area between the rotator cuff and the insertion point of the bone. The method of suture emplacement of the present invention also takes advantage of the thicker bands and central tendons of the rotator cuff because the suture is passed into and across these denser tissue structures which allows for a stronger repair in most cases. Accordingly, a surgeon can approximate the location of the sutures being passed to maximize the core tendons of the rotator cuff to increase strength of the repair.

It is also contemplated with the present invention that ultrasound imaging can be used to aid in the deployment of the needle towards the part of the rotator cuff having the most dense tissue quality. The invention may also reduce cost of a surgical procedure by reducing or eliminating the need for a “double row repair” and the required implants associated with the double row repair because the suture and anchor are more optimally located in the tissue with greater surface areas in contact.

The device of the present invention is also well suited for employment of ‘side-to side’ sutures that are used to close gaps between tendons in a rotator cuff repair. In this scenario, the tendon is grasped and suture is deployed in a similar method without the requirement to use implants. In addition, current suture passing devices often ‘mis-fire’ because the needle is capturing the suture at the most distal portion of the jaws in which needle and suture misalignment can occur. The current invention reduces that risk because the suture can either be loaded before the procedure or is captured at the proximal portion of the jaws.

The device and method of the present invention are especially adapted for use with a barbed suture configuration that allows the surgeon to pass the suture along the alignment of the tissue, but the suture maintains resistance to prevent the suture from being pulled back in the opposing direction after deployment. The barbed suture's resistance in tissue when tensioned also provides the ability to secure the suture to bone and manipulate the tissue during the repair. The unique ability to manipulate the tissue with the device and method of the present invention provides the surgeon a better opportunity to re-approximate tissue to a desired location for an optimal repair. The surgeon is therefore able to pull the rotator cuff in a desired direction from the lateral edge as opposed to having to rely on a single point of suture attachment that is traditionally passed more medial on the rotator cuff. The barbed suture configuration could vary in pattern or rigidity to optimize resistance and strength of the repair. Ultimately, a barbed suture can be of a construction that varies or alternates in thickness to reduce the incidences of suture from being pulled out when deployed. It is contemplated that the barb length, size, pattern, and material can be modified to best match the particular requirements for the procedure to be conducted. Therefore, the present invention allows the suture construct to be optimized for the type of repair and resistance strength required.

The device of the present invention allows the surgeon to secure the tissue, while the subsequent needle advances a barbed like suture through the central portion of the tissue along the axis of the tissue. This orientation of the suture results in placement of the suture to maximize its contact surface with the tissue, yet does not present a barrier between bony contact surface and soft tissue. The device and method also simplifies a rotator cuff procedure because a surgeon is able to deploy the suture without having to retrieve a subsequent suture limb, and therefore the surgeon is not required to tie knots for completing the suture emplacement. Suture management, which is a difficult part of any surgery, is made easier because there are no longer two limbs of suture in the joint space after each suture is passed, which is the result of use of current devices in which the suture is passed in manner requiring the ends of the suture to be secured. The particular employment of the suture according to the present invention may also reduce the burden on the surgeon attempting to maximize the amount of tissue captured by the suture, also referred to as the “tissue bite”, because of the increased strength and tissue density when deploying the suture through the central cross-section of the rotator cuff. Ultimately, the surgeon is better able to manipulate and tension the rotator cuff because the suture is pulling all of the tissue it is in contact with after being passed as opposed to a smaller cross-sectional area of the tissue.

The suture may also incorporate other mechanical characteristics to provide resistance to the suture being pulled in the opposing direction after being advanced. One example includes an implant fixed on the distal end of the suture that changes angles after being advanced to eliminate the suture from being pulled back. In this embodiment, the suture has the implant that is advanced along the axis of the suture, but after the needle is retrieved, the implant changes its orientation to provide resistance from pullout. A small bar or pin is provided that moves from the axis of the suture to approximately perpendicular to the suture after being deployed. Other forms of an anchor may include a button shaped anchor, a ‘T-bar’ anchor, a knot not yet deployed, or any element larger than the overall diameter or portion of the suture itself deployed through the tissue.

The implant can be stored within a sheath or within a cannula of the needle until it is in the desired location, and the removal of the sheath causes the axis of the implant to change thereby placing it in an anchored position to provide pullout resistance. One specific method to deploy the implant includes a needle designed to allow the implant and suture construct to easily be advanced, but then upon removal of the needle, the implant is engaged in the surrounding or adjacent tissue. One way to achieve this method includes providing a needle design with a partial cannula or chamber that holds the anchor. When the needle reverses direction after deployment of the suture, the anchor is removed from its stored position and engages the tissue in a deployed position. Another way to achieve anchor placement includes the use of a sleeve that houses the suture and advances the implant within the needle. In a similar way, the suture could be reoriented resulting in the presentation of a larger surface area of the anchor against the surrounding tissue when the suture is tensioned after being advanced to the targeted location. Therefore when tension is applied, the suture deforms or is reoriented to present a larger profile against the tissue as compared to the body of the suture, and thereby enabling the anchor to remain in place.

The present invention also accommodates the ability to have multiple suture passes when manipulating the handle. In the example of a rotator cuff repair, the needles access the rotator cuff from the lateral edge to the medial aspect, but allow for multiple channels to pass multiple needles in different trajectories. The geometry of the jaw may vary to accommodate multiple needles through independent channels in the shaft that exit the channels in parallel or differing trajectories along the axis of the shaft. Further, the jaw may widen such that the distal end is wider than the proximal end to accommodate the multiple needles. The proximal end has multiple cannula for the needles to deploy. The cannula therefore allow for dual, triple and quad channels of needles that form desired suture deployment patterns. The invention further provides for multiple physical configurations to modify the trajectory of each of the needles to be deployed. Further, the suture may be folded on itself when advanced in these configurations, thereby resulting in two limbs of suture exiting the lateral edge of the rotator cuff.

Because of the pathway or passage orientation of passing a needle according to the invention, the invention allows the geometry of the needle to be modified at the distal end. The present invention can incorporate a sharp tip as opposed to a common blunt tip to allow for percutaneous passing of suture because of the various geometrical pathways provided with the suture passing device of the invention.

Suture passing can also be modified to an angled pass or angle so the suture takes a slight diagonal or oblique approach to the tissue, either inferior to superior or vice versa. The needle in this angular pass still makes access to the tissue towards the proximal end of the jaws, but uses a diagonal pathway to capture a cross section of the rotator cuff. In this embodiment, either a barbed type suture configuration may be used, or a suture construction that includes an anchor as described above in which the anchor exits the rotator cuff at the most distal end of the deployment to eliminate the suture from being pulled in the opposing direction. In this modified method of suture passing, a surgeon is still provided the advantage of suture passed as described previously, but is also provided with the possibility of increasing the strength of the suture's resistance from being pulled out with an implant that exits the tissue at some point in deployment.

Because the device can be used in conjunction with a cannulated needle that contains the suture and an implant, as opposed to a traditional flat, non-cannulated cutting needle, a needle incorporates cannula oriented to a center or main axis of the tissue being repaired. The method of introducing the needle in this invention for suture passing further accommodates healing agents (such as PRP or other factors) to be administered directly and simultaneously during suture passing. This is particularly advantageous for diseased tissue to help optimize the results of the repair. The needle, for example, could be fenestrated in a manner to facilitate a healing agent injected into the needle also being used for suture passing. Designing a luer lock or connecting feature common to syringes on the proximal end of the needle being deployed conveniently allows the introduction of a healing agent into the tissue and repair site.

The invention could also be used as a standalone needle passing device that allows for a needle to access a cross-section of tissue for introduction of injections specific to the pathology encountered. Current needles cannot consistently and accurately access desired tissue and are commonly just injected into the joint space and thus, diluting the impact of the agent.

The present invention can also be modified to load the suture in the distal end of the grasping jaw. In this way, the needle passes from lateral to medial, as set forth in the previously described method, but the needle captures the suture at the distal end of the jaw and thus pulls the captured suture from medial to lateral when the needle is retrieved. In this way, the needle is deployed without suture, however the needle is advanced to the distal end of the jaw to capture the suture upon full deployment of the needle. The suture is then retrieved or pulled back through the center of the tissue that the needle has taken upon deployment.

The present invention also allows the user to utilize image guided imaging technology in conjunction with rotator cuff repair, such as ultrasound imaging. The ability to access and insert a needle in the lateral edge of the rotator cuff enables the surgeon to couple the invention with image guided technologies to visualize the ideal pathway to pass suture. The surgeon can evaluate tissue quality by the density shown in the image, and then grasp the optimal portion of tissue. The invention further enables the surgeon to utilize the differing bands and thickness of the rotator cuff when passing suture. The strength of the repair is stronger through optimizing the tissue and suture construct. According to a simplified explanation of this method of the invention, a surgeon follows the following general steps: (1) using an image guided technology to visualize the rotator cuff tendon to be repaired; (2) observing differences in tissue density and strength; (3) identifying the major “bands” or the central tendon which may optimally receive the suture; (4) grasping the desired portion of the tissue based on the images; and (5) deploying the needle from lateral to medial that results in passing the suture into the desired thicker portion of the tendon. It should therefore be apparent that the above described device and method allows the surgeon to utilize ultrasound and a percutaneous approach of the needle and suture to minimize the invasiveness of the surgery.

The present invention gives the surgeon an opportunity to maximize the chances of a successful repair by taking advantage of the existing anatomy of the rotator cuff by suture emplacement in the lateral to medial orientation. As mentioned, the above described methods can be utilized to secure other soft tissues to bone by modifying the grasper size and configuration to tension the tissue. For example in a meniscus repair, the needle is still deployed in the same manner to increase the potential strength and ease of use for the surgeon. More specifically, the grasper of the device can be scaled in size appropriately to be used in a meniscus repair. The meniscus is grasped and the needle is deployed in the same manner across the tear. An implant can also be used to anchor the suture(s) and to provide tension across the tear. This device and method may eliminate the need for a “back-stop” type anchors or knots on the surface of the meniscus used in current devices. Current devices involve needles and other penetrating devices to deploy an implant or suture. With these current devices, often times the meniscus can turn or deform while applying pressure. The present invention stabilizes the meniscus while deploying a needle across the tear and utilizing one of the described implant options to provide optimal resistance and fixation.

With respect to some of the features or characteristics of the invention, it is contemplated that the invention may include the following features. It however should be understood that these features are not the only ones that can be adopted in the preferred embodiments, and further, these features can be adopted alone or in combination with one another in one or more preferred embodiments. These features include: 91) a jaw type configuration that grasps the desired tissue (2) a handle that allows a user to grasp and/or lock in a ratcheting component of the handle to secure the rotator cuff or other tissue involved in a procedure; (3) a handle that allows the user to deploy a needle along the axis of the rotator cuff from lateral to medial (4) a jaw construction designed to deploy the needle through the most central or thickest portion of the rotator cuff and to advance the needle medially (5) a jaw construct that advances a needle in between the opposing jaws (6) a needle that advances suture along the axis of the jaw or shaft and through the described central portion of the tissue (7) a needle that enters the central portion of the tissue but may also incorporate a slight angle to deploy the needle above or below the rotator cuff so that further resistance can be applied (8) a suture designed to provide resistance when tensioned in the opposite direction it was introduced (9) a handle and jaw design that allows for single or multiple passes of suture (10) a handle and jaw design that accommodate multiple channels to house each needle being deployed (11) a handle and jaw design that allows for the needle or suture to be passed at different angles through the central portion of the rotator cuff (12) a jaw construct customized to address the same passing technique for other soft tissue structure, such as scaling the size of the jaw for meniscal repair (13) a suture passing method that integrates ultrasound during rotator cuff surgery to optimize the repair via selecting tissue so the suture to rotator cuff construct is maximized (14) a suture passing method and device that allow for percutaneous and less invasive rotator cuff repair (15) a needle to allow for the injection of healing agents (PRP, Botox, or any desired agent). Perforations, fenestrations, or a standard beveled needle could be used; however a fully or partially cannulated needle could be used to optimally access the desired cross section of targeted tissue. Alternatively, a needle that has a standard syringe attachment on the proximal end can be used to accommodate introduction of agents.

The invention is especially adapted for use in rotator cuff repair. However, the invention is not limited to only this type of tissue repair, and can be used for other repairs or reattachment of tendon, muscle, ligament, or meniscus. The invention generally relates to a method and device utilized to pass suture into the desired soft tissue by grasping the desired tissue, deploying a needle and advancing the suture along the axis of the shaft. The needle enters and is advanced in or near the center of the targeted soft tissue from the lateral to medial directions or areas.

The invention also includes a suture construct that provides resistance from being pulled in the opposite direction once it has been advanced. The suture construct may have a barb type or other similar configuration that helps to prevent the suture from being pulled back out of the soft tissue after being advanced. In this invention, “suture” is intended to cover any material commonly used in soft tissue repairs and includes, but is not limited to, braided suture, woven suture, PDS, monofilament, quill or any braided or non-braided construct of varying thickness or size used to tension, tie, or secure soft tissue. The term suture is also intended to include barbed like configurations or other constructs that may be attached to the suture to prevent the suture from migrating in the opposing direction after being advanced in the tissue.

The invention can also be defined with respect to various aspects or embodiments of the invention. These aspects or embodiments include the following, it being understood that these are not to be interpreted as the only aspects or embodiments of the invention, as it will be appreciated that there are other aspects and embodiments of the invention that may also be defined based upon the figures and written description herein. In a first aspect of the invention, it may be considered a suture passing device, comprising: (1) a pair of handles; (2) a shaft operably connected to the handles; (3) a trigger mechanism operably connected to the shaft; (4) said shaft having a distal end including a stationary jaw and a movable jaw controlled for movement between and open position and a closed position by manipulating said handles; (5) said shaft having an opening there through to receive a suture passing needle and attached suture; (6) said shaft extending along a longitudinal axis; and (7) wherein said needle being advanced by operating the trigger mechanism in which the needle is advanced in a direction parallel to the longitudinal axis of the device through an opening in the shaft and into targeted tissue. Further aspects of the device include said opening of said shaft having a plurality of openings extending substantially parallel to one another through said shaft, thereby enabling a corresponding plurality of suture passing needles to be passed simultaneously into targeted tissue by operation of said trigger mechanism.

In another aspect of the invention, it may be considered a method of passing suture into targeted tissue, said method comprising: (1) providing a suture passing device, comprising: (a) a pair of handles; (b) a shaft operably connected to the handles; (c) a trigger mechanism operably connected to the shaft; (d) said shaft having a distal end including a stationary jaw and a movable jaw controlled for movement between and open position and a closed position by manipulating said handles; (e) said shaft having an opening there through to receive a suture passing needle and attached suture; (f) said shaft extending along a longitudinal axis; (2) advancing the needle through the device by operating the trigger mechanism; and (3) wherein the needle is advanced in a direction parallel to the longitudinal axis, and through the shaft into the targeted tissue. In yet another aspect of the invention, it may be considered a method of passing suture into targeted tissue for rotator cuff repair, said method comprising: (1) providing a suture passing device, comprising: (a) a pair of handles; (b) a shaft operably connected to the handles; (c) a trigger mechanism operably connected to the shaft; (d) said shaft having a distal end including a stationary jaw and a movable jaw controlled for movement between and open position and a closed position by manipulating said handles; (e) said shaft having an opening there through to receive a suture passing needle and attached suture; (f) said shaft extending along a longitudinal axis; (2) advancing the needle through the device by operating the trigger mechanism; and (3) wherein the needle is advanced in a direction parallel to the longitudinal axis, and through the shaft into the targeted tissue of the rotator cuff, near a center of mass of the rotator cuff and extending medial to lateral through a thickness of the rotator cuff.

In yet another aspect of the invention, it may be considered, in combination, a suture and anchor repair system, especially adapted for repair of torn muscles or tendons, said combination comprising: (1) at least one suture extending through a thickness of a targeted muscle/tendon, and in a direction substantially parallel to a direction of extension of the targeted muscle/tendon; and (2) at least one anchor secured to a bone structure adjacent the targeted muscle/tendon, and said at least one suture attached to said anchor.

In yet another aspect of the invention, it may be considered a suture construction comprising a length of suture, and a plurality of barbs or protrusions extending angularly away from a longitudinal axis of said length of suture, said barbs or protrusions being spaced from one another along a length of said length of suture.

In yet another aspect of the invention, it may be considered a method of deploying an anchor used with a length of suture for preparing targeted tissue, said method comprising: (1) advancing the suture and anchor through a suture passing device, wherein the suture and anchor are passed into the tissue in a direction substantially parallel to a longitudinal axis of a shaft of the suture passing device; (2) placing the suture and anchor at a desired location within the targeted tissue; (3) placing tension on the suture in a direction opposite to the direction of advancement; and (4) rotating the anchor to be disposed substantially parallel to the direction of extension of the suture.

In yet another aspect of the invention, it may be considered a method of deploying an anchor used with a length of suture for preparing targeted tissue, said method comprising: (1) advancing the suture and anchor through a suture passing device, wherein the suture and anchor are passed into the tissue in a direction substantially parallel to a longitudinal axis of a shaft of the suture passing device; (2) placing the suture and anchor at a desired location within the targeted tissue; and (3) removing the anchor from within the shaft, wherein the anchor moves from a stored position within the shaft, to a deployed position within the targeted tissue, the deployed position resulting in expanding at least one dimension of the anchor.

In yet another aspect of the invention, it may be considered a suture passing needle, comprising a linear extending needle shaft having an opening extending through said shaft, said shaft having a sharp distal tip, and a notch formed in the distal tip. This aspect may further include said needle having a plurality of perforations extending through said needle shaft.

In yet another aspect of the invention, it may be considered, in combination, a system for deploying an anchor and suture into targeted tissue, said system comprising: (1) a shaft of a suture passing device having an opening extending through said shaft; (2) a deployment sleeve extending through said opening; (3) a length of suture extending through said sleeve, and having a distal end extending beyond a distal end of said sleeve; (4) and an anchor attached to the distal end of said suture.

### BRIEF DESCRIPTION OF THE DRAWINGS

FIG. 1 is a side view of a conventional suture passing device;

FIG. 2 is another side view of the device of FIG. 1, when the device is grasped by a user in a first position to manipulate the jaws of the device;

FIG. 3 is another side view of the device of FIG. 1, when the device is grasped by a user and a second position to deploy a suture needle;

FIG. 4 is an enlarged view of the jaw at the distal end of the device in a closed position;

FIG. 5 is another enlarged view of the jaw, but with a needle deployed in which the needle is oriented in a perpendicular orientation with a long axis of the device;

FIG. 6 is yet another enlarged view of the jaw, the jaw being shown in an open position;

FIG. 7 is a perspective view of the device used during a surgical procedure, namely, repair of a rotator cuff injury in which the jaw of the device is shown in a position to grasp a rotator cuff, and in a second view with the jaws in a closed position grasping the rotator cuff;

FIG. 8 is another perspective view of the device shown with the jaws in a closed position when grasping a rotator cuff;

FIG. 9 is an enlarged perspective view of the distal end of the device showing loading of a suture in a suture passing method;

FIGS. 10A-10D illustrate preferred embodiments for jaw designs of the present invention;

FIG. 11A illustrates a schematic diagram of the suture of the present invention as it approaches a targeted tissue, such as a tendon or muscle;

FIG. 11B illustrates the jaws of the device grasping the targeted tissue, and an axis at which the needle will be passed into the tissue;

FIG. 11C illustrates the device with a different jaw configuration, and multiple channels or cannulations that enable passing of multiple needles in distinct directions;

FIG. 12 illustrates the device having the multiple channels or cannulations;

FIG. 13 illustrates the direction of a needle for repair of a rotator cuff tendon that extends lateral to medial and through the main axis or thickness of the tendon;

FIGS. 14 and 15 illustrate example suture configurations for a rotator cuff repair;

FIG. 16 is an example of a prior art suture configuration;

FIG. 17 illustrates optimal orientations for multiple needles that may be used to pass suture into a targeted tendon;

FIGS. 18A-C illustrate further examples of suture and anchor configurations in a repair;

FIG. 19 is a preferred embodiment of a barb type suture;

FIGS. 20A-C illustrate examples of use of the barb type suture in a rotator cuff repair;

FIGS. 21 A-B illustrate an example of a “T-bar” anchor;

FIGS. 22A-B and FIGS. 23A-B illustrate various anchor and suture arrangements in a rotator cuff repair;

FIG. 24A illustrates another preferred embodiment for an anchor, showing the anchor in a folded or stored position;

FIG. 24B illustrates the anchor of FIG. 24A in a spread or deployed position;

FIGS. 25A-B illustrate use of the anchor of FIGS. 24A-B in a rotator cuff repair;

FIG. 26A illustrates a knot-bundle type anchor in a stored or compressed position;

FIG. 26B illustrates the knot-bundle type anchor in an expanded or deployed position;

FIG. 27A illustrates a preferred embodiment for a suture passing needle having a cut-out or notch to hold suture;

FIG. 27B illustrates another preferred embodiment for a needle having a cut-out or notch to hold suture;

FIG. 28 is another preferred embodiment for a suture passing needle having a slot formed in the distal end for frictionally holding an anchor;

FIG. 29 illustrates an anchor and suture stored within the interior channel of a needle;

FIG. 30 illustrates a pair of anchors and suture stored within the interior channel of the needle;

FIG. 31 illustrates an anchor with one end or tip held within the slot of the needle;

FIG. 32 illustrates a deployment sleeve used to assist in placing an anchor and suture;

FIG. 33 illustrates the anchor of FIG. 24A in the stored position within the needle;

FIG. 34 illustrates the anchor of FIG. 33 in the spread or deployed position;

FIG. 35 illustrates another preferred embodiment for a suture passing needle having a distal slot that is longer than the slot illustrated in the FIG. 28;

FIG. 36 illustrates a cross-section of the needle of FIG. 35, and showing and anchor disposed within the slot:

FIG. 37 illustrates one characteristic of a needle, namely, that the needle is flexible;

FIG. 38 illustrates a needle with perforations allowing delivery of medications or therapeutic agents;

FIG. 39 illustrates the needle of FIG. 38 attached to a syringe;

FIG. 40 illustrates the suture passing device as it approaches another type of tissue, namely, a meniscus;

FIG. 41 illustrates one preferred orientation for suture to be passed through the meniscus to repair a tear;

FIG. 42 illustrates a barb type suture used for repair of the tear;

FIG. 43 illustrates use of a “T-bar” type anchor and suture used for repair of the tear; and

FIG. 44 illustrates use of the “T-bar” anchor and suture, however, the anchor is located exteriorly of the meniscus.

### DETAILED DESCRIPTION

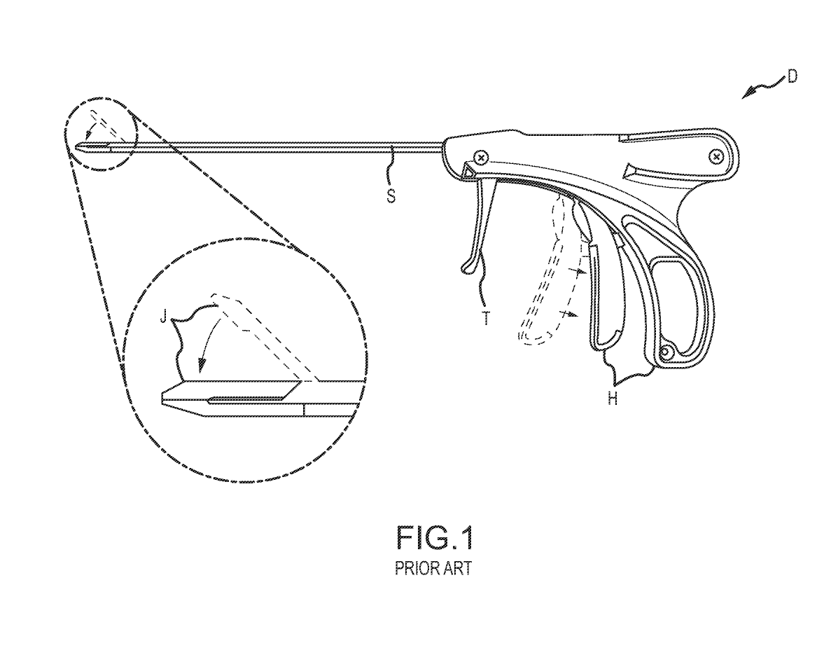

FIGS. 1 through 11 illustrate a prior art suture device. Referring first to FIG. 1, the prior art device D is characterized by a pair of handles H, a tubular extension or shaft S, and a separate trigger mechanism T. Jaws J are located at the distal end of the shaft S. The handles are used to manipulate jaws J in either an open or closed position. As shown in the enlarged portion of FIG. 1, the upper jaw rotates up or down to place the jaws in the open or closed positions. The handles H therefore give the user the ability to grasp tissue, as well as to deploy a suture passing needle when the jaws are closed.

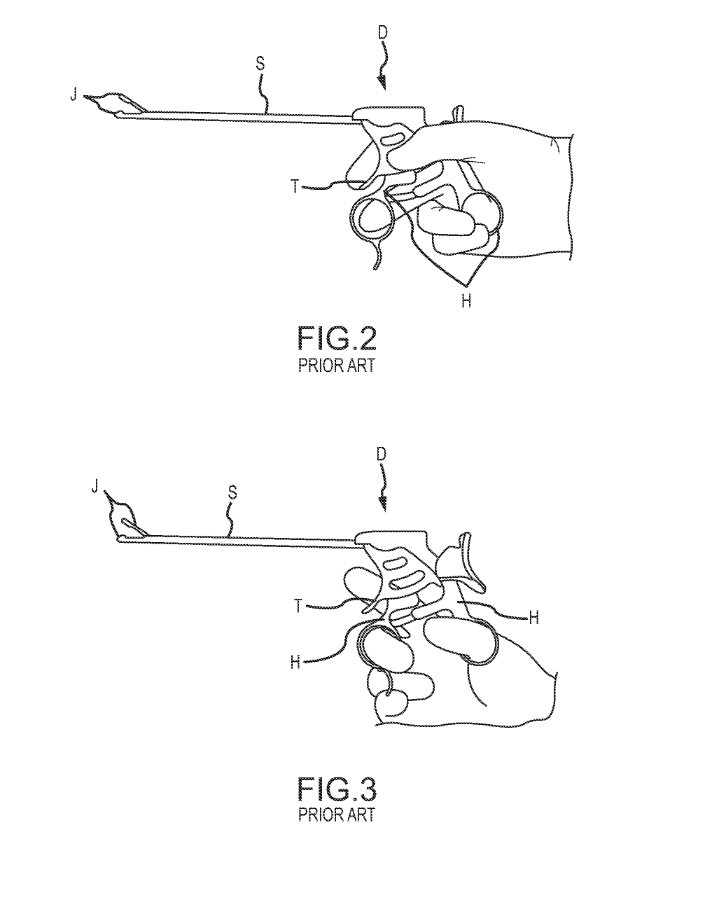

In FIG. 2, the hands of the user are placed in one possible position, especially adapted for firing a needle (not shown) loaded in the shaft S by manipulating the trigger mechanism T with the index finger.

FIG. 3 illustrates another possible hand position in which the user may either manipulate the handles H for opening/closing the jaws J, or fire the needle (not shown) loaded in the shaft S by manipulating the trigger mechanism T with the index finger. The device illustrated in the FIGS. 1-3 is similar to the device disclosed in the U.S. Pat. No. 7,166,116, this US patent being hereby incorporated by reference in its entirety for purposes of disclosing a prior art tissue grasping and suture passing device.

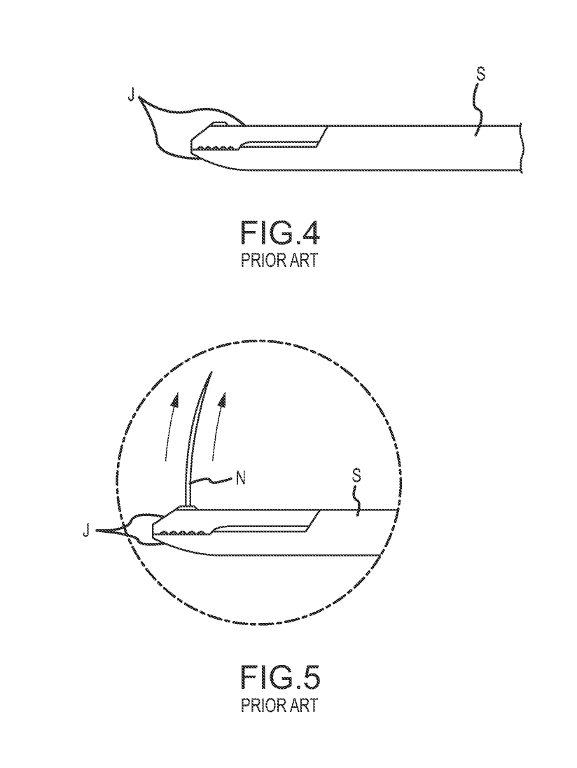

FIGS. 4 and 5 are enlarged views of the distal end of the prior art device with the jaws in the closed position. The particular geometry of the jaws shown in these figures are common to jaw patterns found in many commercial devices. However, it shall be understood that the particular jaw design for the suture passing device of the present invention may vary with respect to any of the jaw parameters to include, without limitation, the jaw length and width, the jaw teeth design, and other parameters. FIG. 5 more particularly illustrates the perpendicular orientation of a suture passing needle N with respect to the longitudinal or long axis of the shaft S. As explained below, the suture passing device of the present invention substantially differs in that it provides a needle pathway after the jaws of the device are closed such that the needle is advanced in a direction parallel or co-linear with the shaft of the device.

FIG. 6 illustrates the movable upper jaw J of the prior art device in the open position. The open position is used when the surgeon is preparing to grasp the desired tissue such as a tendon. When the targeted tissue is grasped, the needle is then deployed from the stationary or lower jaw upwards, through the upper or movable jaw (inferior to superior). As also explained below, the suture passing device of the present invention does not require an opening or slot to be formed in the upper jaw because the needle does not pass through the upper jaw. Therefore, the present invention may adopt an upper and lower jaw with smaller profiles, to include sharp tipped jaws or other configurations that may be procedurally beneficial.

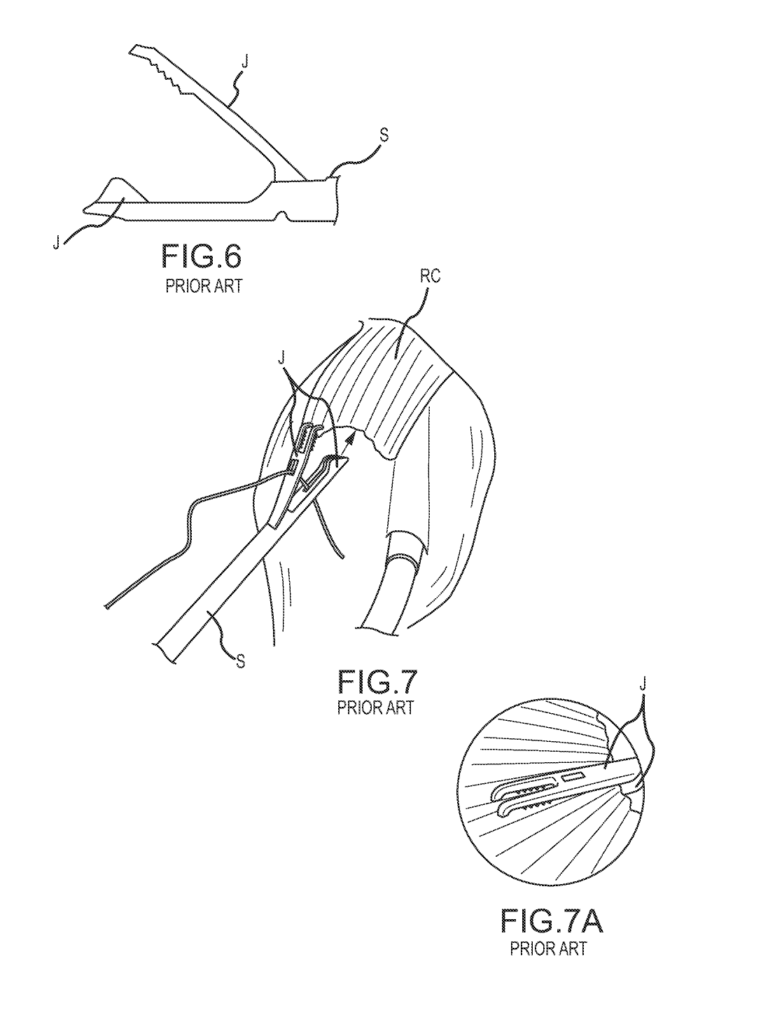

Referring to FIG. 7, the prior art device is shown with the movable jaw in the open position, and the device is prepared to grasp a tendon, such as the rotator cuff RC. As shown in the enlarged area of FIG. 7A, the upper jaw resides above the targeted tendon, and the lower jaw resides below the targeted tendon.

Referring to FIG. 8, the prior art device is positioned to grasp the rotator cuff tendon RC, and the jaws J are in the closed position. FIG. 8 also illustrates that the needle N has been deployed to pass a suture SU through the top of the rotator cuff tendon RC. As shown, the suture is passed from inferior to superior and therefore, perpendicular to the fibers of the tendon. With the device shown in FIG. 8, there are two limbs or ends of suture SU leaving the respective cannula (one from the inferior side and one from the superior side of the rotator cuff). In the present invention, the procedure as explained below is simplified because there are not two limbs of suture that require tying or that extend beyond the medial aspect of the rotator cuff.

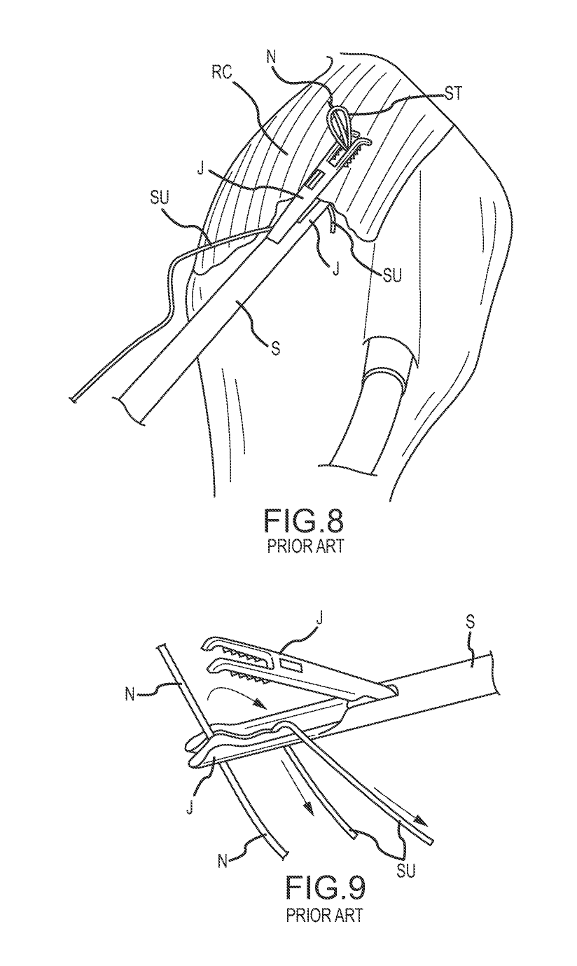

Referring to FIG. 9, this figure shows the loading of a conventional suture passing needle N in the prior art device. This loading is common in many procedures to allow a needle to capture the suture SU when deployed from the bottom jaw through the top jaw of the device. The suture SU is loaded through the distal jaw prior to inserting the instrument into the cannula. As explained below, the suture passing device of the present invention can be loaded at the proximal end of the device, or pre-loaded with the suture and therefore does not require that the needle catch or secure the loaded suture at the distal end of the jaws.

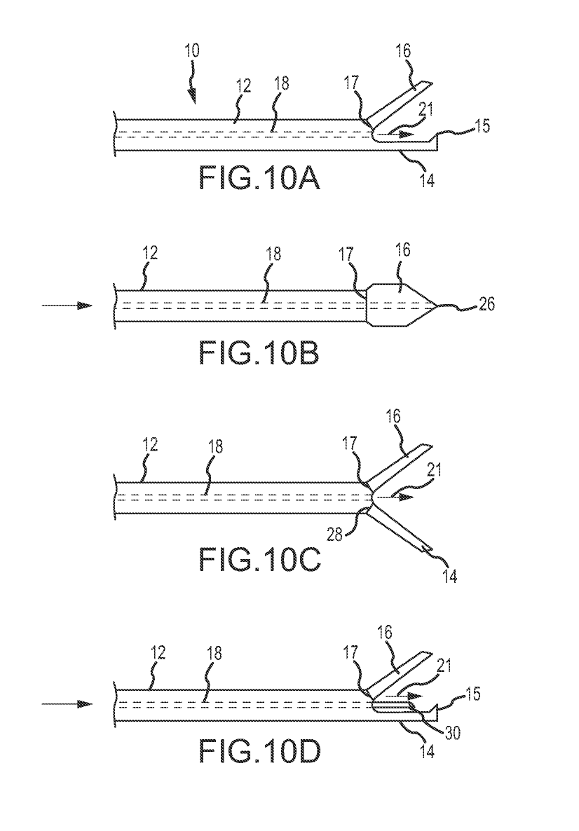

Referring to FIGS. 10A-10D, these figures show preferred embodiments for jaw designs of the suture passing device of the present invention. Because the present invention does not require a gap or slot to receive a needle in the upper jaw, there are greater design options for not only the upper or movable jaw, but also the lower or stationary jaw. As shown in FIG. 10 A, the device 10 includes a shaft or tubular extension 12, and a lower jaw 14 and an upper jaw 16 disposed at the distal end of the shaft 12. A passageway or cannulation 18 extends through the shaft, and which receives a suture passing needle and suture (not shown). The passageway 18 therefore enables the needle to travel in the parallel or co-linear direction as shown by the directional arrow 21. The lower jaw 14 in FIG. 10A is a substantially linear extension of the tubular extension 12, and further includes a distal flange 15. The upper jaw 16 is movable, that is, rotatable about it hinge point or hinge axis 17.

FIG. 10 B, is a plan view of the device of FIG. 10A. The channel or passageway 18 extends along the axis or direction of extension of the shaft 12. As also shown, the upper jaw 16 has a pointed tip 26, and does not have a slot for gap for receiving a needle.

Referring to FIG. 10C, in yet another embodiment, the device 10 has both the upper and lower jaws that can be articulated. Accordingly, the lower jaw 14 also has a hinge point or hinge axis 28 about which the lower jaw may rotate. In order to achieve this dual articulation, instead of utilizing just a single control cable (not shown) for manipulating the upper jaw, an additional control cable (not shown) can be used for operating the lower jaw 14.

Referring to FIG. 10D, this figure illustrates a needle 30 that has passed through the passageway 18, and in a position for entry into the targeted tissue. More specifically, the needle 30 exits the shaft 12 at a trajectory or angle that is parallel with the axis of the device, and that also may be substantially parallel with the long axis of the rotator cuff tendon (illustrated in later figures). Therefore, the needle 30 is able to take a pathway through the rotator cuff tendon near the center of mass and the main axis of the tendon, thus extending medial to lateral through the thickness of the tendon.

It should be understood that FIGS. 10A-10D are schematic diagrams, and are intended to show the general shape of the jaws and functioning of the jaws. These figures are not necessarily drawn to exact scale or shape.

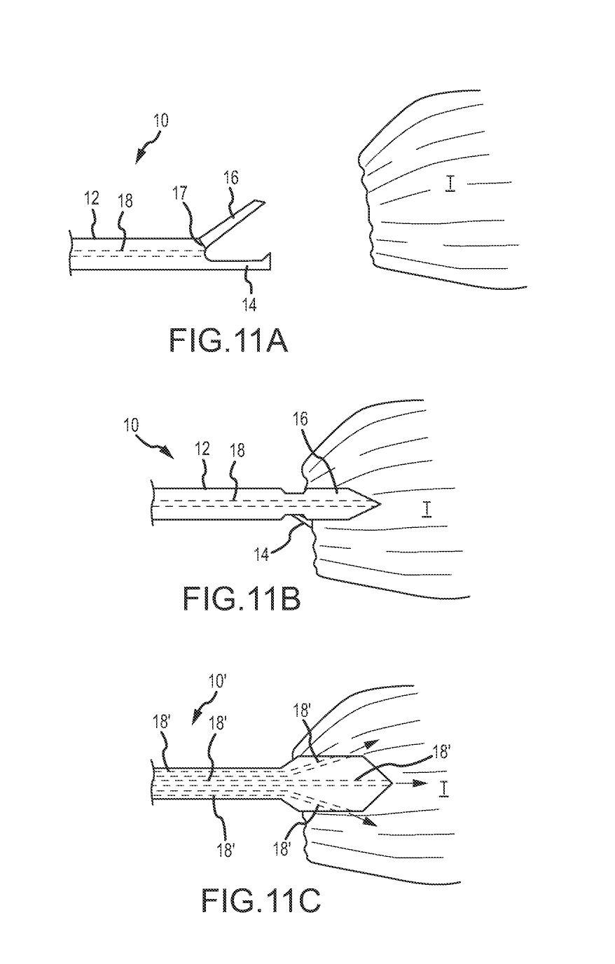

FIG. 11A illustrates the device 10 as it approaches a targeted tissue T, such as the rotator cuff. FIG. 11 B illustrates the device 10 as it is advanced toward the tendon, and with the jaws moved to a closed position in order to grasp the tendon. In FIG. 11 B, the dotted line represents the passageway 18, and also the directional pathway of the needle (not shown) that passes through the tendon.

In yet another embodiment, FIG. 11 C represents a device 10′ that has multiple channels or passageways formed through the shaft 12 which therefore provide the user with the ability to directionally control the advancement of the needle in multiple different directions. This additional functionality is achieved by providing multiple channels 18′ through the shaft in which each of the channels may be loaded with corresponding suture passing needles. The additional channels may vary in number and diameter to receive different types of suture needles. Further, some of the needles may have a curvature, such as provided by a needle made of pre-shaped nitinol. When a curved, pre-shaped needle exits the channel in which it is loaded, it will therefore pass into the targeted tissue along the pre-designated trajectory which therefore allows optimal deployment of the needle and suture.

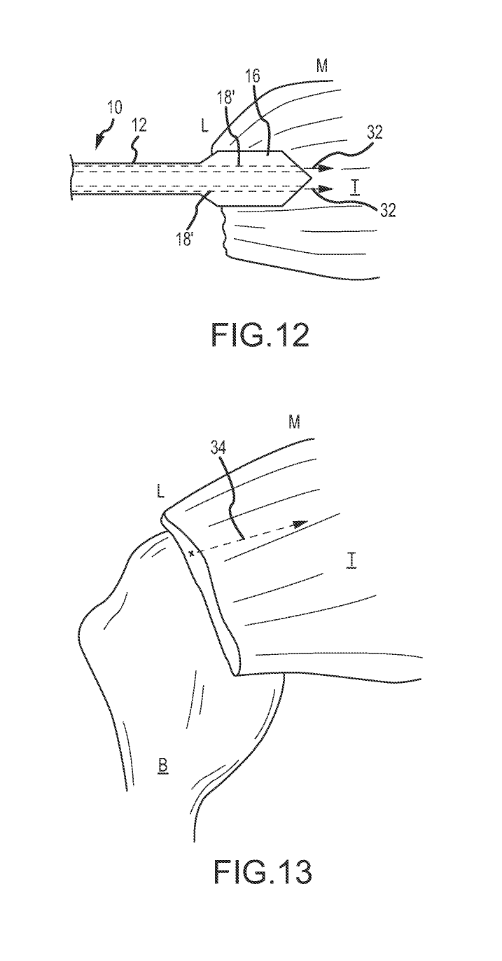

FIG. 12 shows yet another embodiment of the device 10 in which two channels 18′ are provided to receive and pass two corresponding suture needles and corresponding sutures (not shown) simultaneously. The directional arrows 32 illustrate the general trajectories or directions in which the needles are deployed. The needles are deployed from lateral L to medial M as the tendon T is oriented in the figure.

FIG. 13 illustrates the pathway 34 that the needle and attached suture (not shown) take through the tendon T. What is intended to be illustrated in this figure is again a rotator cuff tendon T shown adjacent the bone structure B. The pathway 34 clearly shows the ability to locate a suture through the thickness of the tendon T beginning at the lateral edge, and then extending through the tendon thickness medially along a selected length thereof. The tissue mass that is in contact with the suture is maximized and therefore provides a much better manner in which to anchor the tendon for an optimum tendon repair.

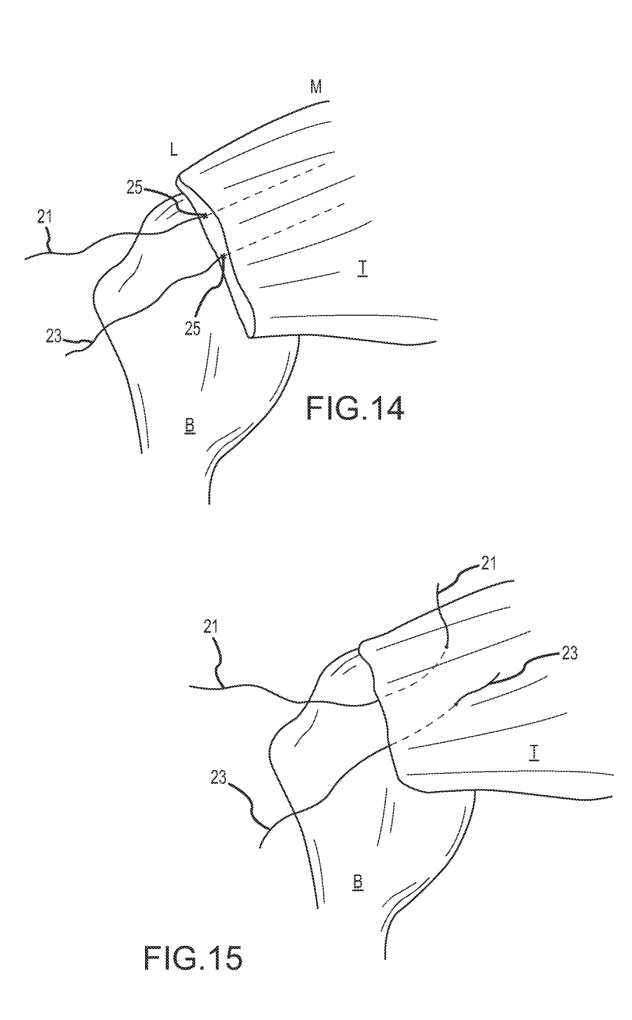

Referring to FIG. 14, this figure illustrates an example of two sutures that have been located within the rotator cuff, namely, suture 21 and suture 23. This figure also illustrates the sutures 21 and 23 being passed from lateral to medial. As also shown, the entry points 25 of the needles pass through the center of mass of the thickness of the tendon T, which greatly enhances the ability of the sutures to hold the tissue.

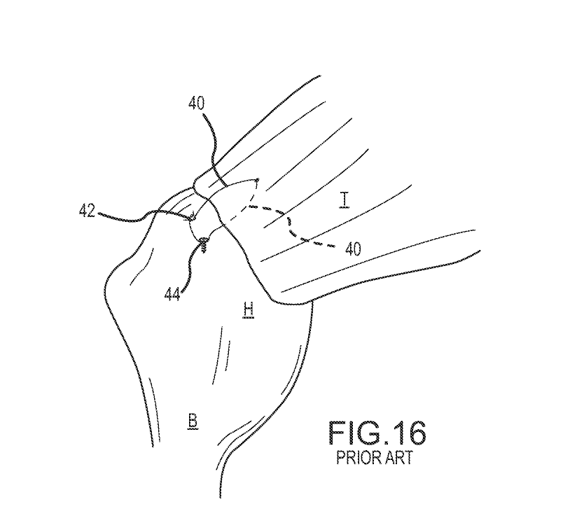

FIG. 15 represents placement of the sutures 21 and 23, and the limbs or free ends of the sutures that remain after the needles have passed through the tissue. Thus, the FIG. 15 illustrates an example in which the needles pass completely through the tendon T, while the needles do not pass completely through in the FIG. 14.

FIG. 16 represents a traditional suture arrangement for repair of a rotator cuff What is intended to be shown in this figure is that a suture 40 first passes below or under the tendon T, and then is passed vertically through the tendon (inferior to superior), and exits through the top surface of the tendon T. The suture 40 is then tied down to secure the tissue resulting in a knot 42 that ties the two ends of the suture together. The suture is typically secured to an implant or anchor 44. As is evident in the comparison between the FIGS. 15 and 16, is that the prior art technique in FIG. 16 results in very small mass of the tissue that is actually is in contact with the suture, namely, the confined cross sectional area through which the suture is passed in the inferior to superior direction. Therefore, the suture is much more prone to moving or tearing through the tissue which can certainly compromise the success of the procedure. Also, the amount of contact surface is reduced with respect to the humeral head H.

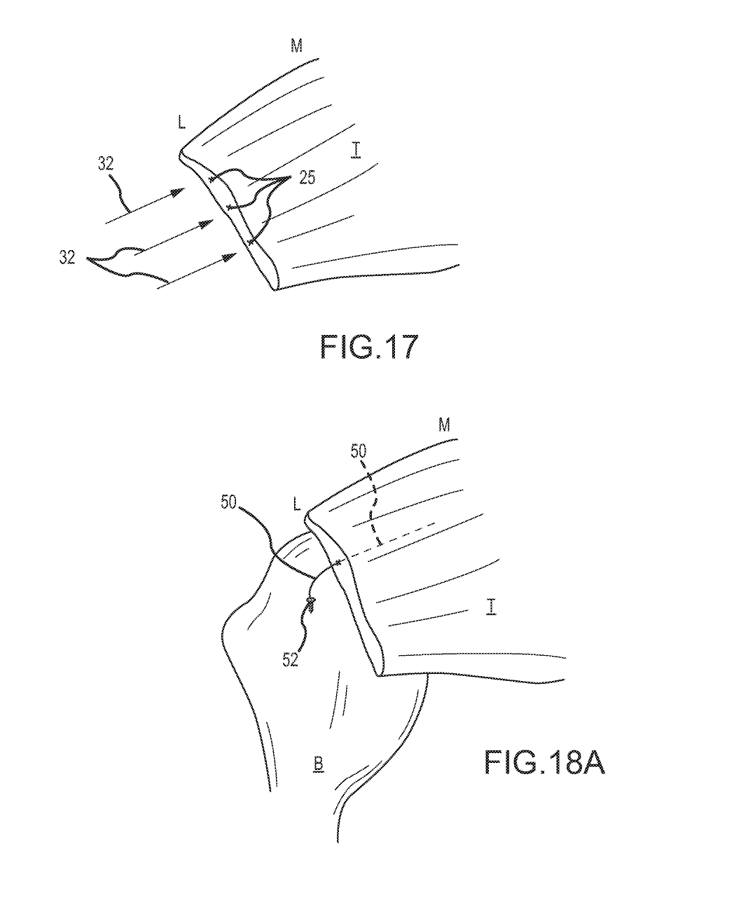

FIG. 17 illustrates possible approach paths for the sutures, again referencing the directional arrows 32. Also, this figure shows specified entry points 25 through the thickness of the tendon that can be targeted for entry and passing of the sutures.

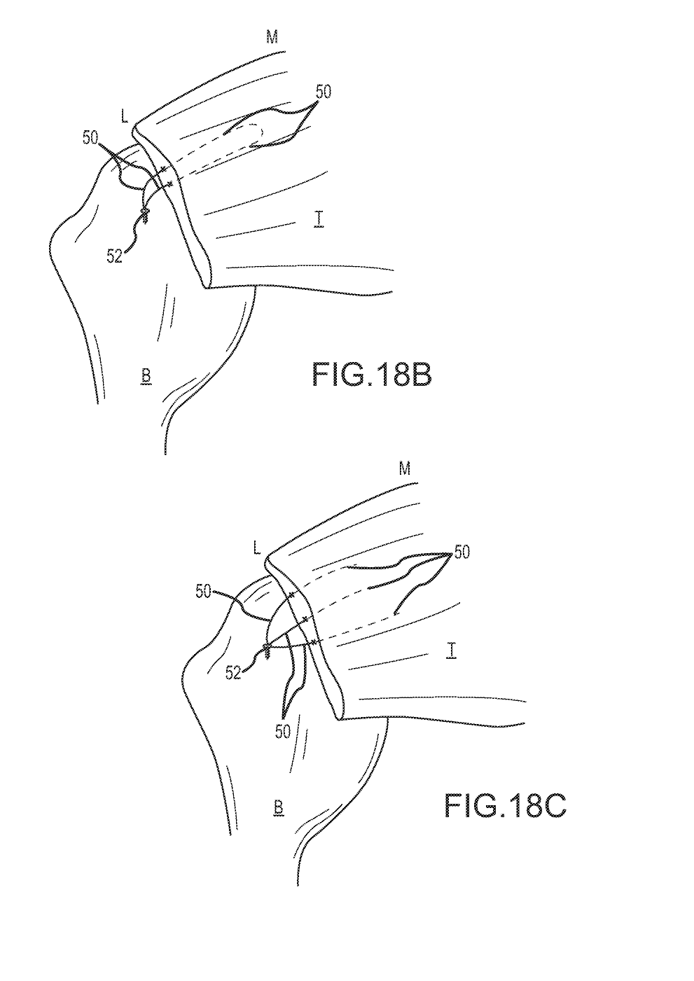

FIG. 18A shows a single suture 50 deployed into the center of mass or center cross section of the rotator cuff tendon T, noting that there is only one free end or limb that is exposed and secured to an anchor 52, the other end of the suture being located within the tendon, and therefore not requiring separate anchoring or passing back through the tendon for tying to the opposite end thereof. Accordingly, with the use of a single suture in the present invention, a knot arrangement is not required. The suture is therefore used in tension to connect the tendon T to an implant 52 of the surgeon's choice. As shown in the FIG. 18B, the suture 50 is passed through the tendon T in a U-shaped configuration and therefore, the suture as deployed has two limbs or free ends. These free ends or limbs can each be secured to the implant 52 as shown. Therefore, with a single suture, an extremely high surface area contact can be achieved with the suture, again avoiding mold multiple sutures, multiple knots, thereby simplifying the procedure.

FIG. 18C illustrates other potential suture arrangement in which multiple sutures 50 are used, and each are secured to a single implant 52. Therefore, one can appreciate the great number of suture arrangements that are made available to a surgeon in a simplified suture passing method of the present invention.

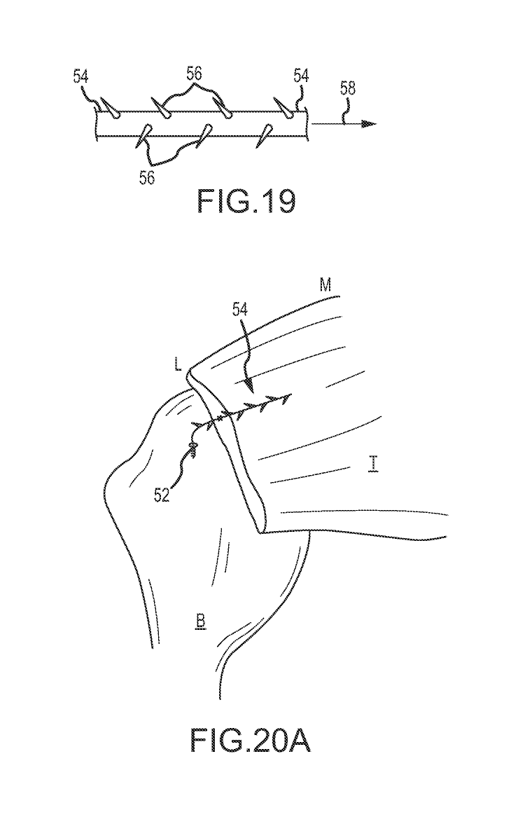

FIG. 19 represents a suture 54 in accordance with another embodiment of the present invention. The suture 54 has a plurality of barbs or protrusions 56 that are spaced from one another along the length of the suture. This suture construction allows the suture to enter tissue in the direction that the needle is deployed, but provides resistance to pull-out in the opposing direction. More specifically, FIG. 19 shows the suture 54 and the barbs 56 extending at an obtuse angle with respect to the direction 58 that the needle (not shown) is deployed. The protrusions 56 will slightly bend or fold against the suture body during placement, which therefore enables the suture to be deployed with the needle. However, once the suture is placed, it is highly resistant to pull-out in the opposite direction, since the barbs or protrusions 56 become embedded within the surrounding tissue. The barbs can vary in size, rigidity, number, and the designated plane in which they are designed to maximize resistance from pull-out. In the case of a rotator cuff repair, the barb type suture would be deployed lateral to medial, thereby preventing pullout from medial to lateral. Ultrasound imaging could be used to identify the densest bands of tissue for deploying the barbed suture. Therefore, the barbed suture located in the densest tissue bands would increase the strength of the repair.

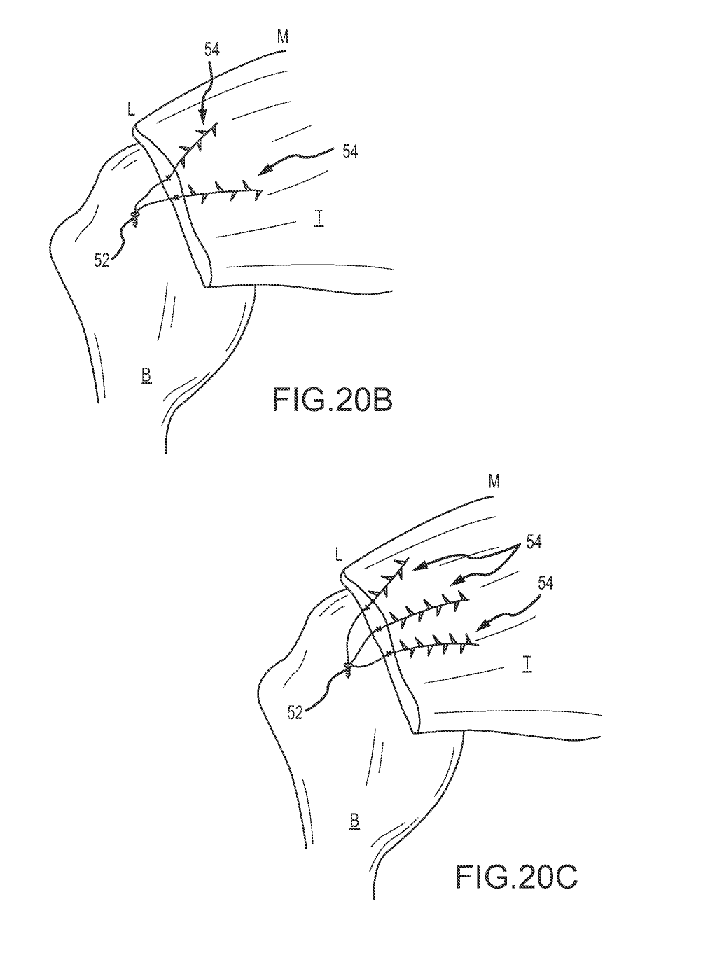

FIG. 20A illustrates one example of the suture 54 deployed for repair of a rotator cuff using a single suture 54. FIG. 20 B illustrates the use of two barb type sutures 54, while FIG. 20 C illustrates the use of three barb type sutures 54. These figures illustrate the ability of a surgeon to pull, tension, and re-approximate the rotator cuff to the desired location for an effective repair. These figures illustrate repair in conjunction with use of a single implant; however, it shall be understood that the surgeon has the capability to deploy one or more implants.

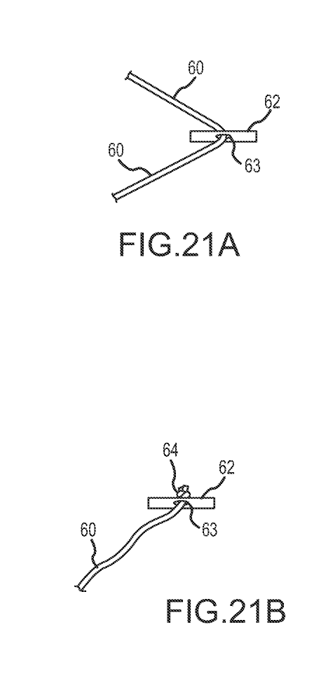

Referring to FIGS. 21A and 21B, a length of suture 60 is illustrated, along with an implant 62 that can be used to provide additional resistance to pullout of the suture when deployed. The implant 62 can be a selected length of flexible material, with an aperture 63 that receives the suture 60. One end of the suture 60 may include a knot 64, the knot being larger than the aperture 63. The suture 60 shown in FIG. 21B is ready for loading within the cannula of the suture passing needle (not shown) in which the length of the implant 62 remains parallel and loaded within the cannula for easy placement within the tissue.

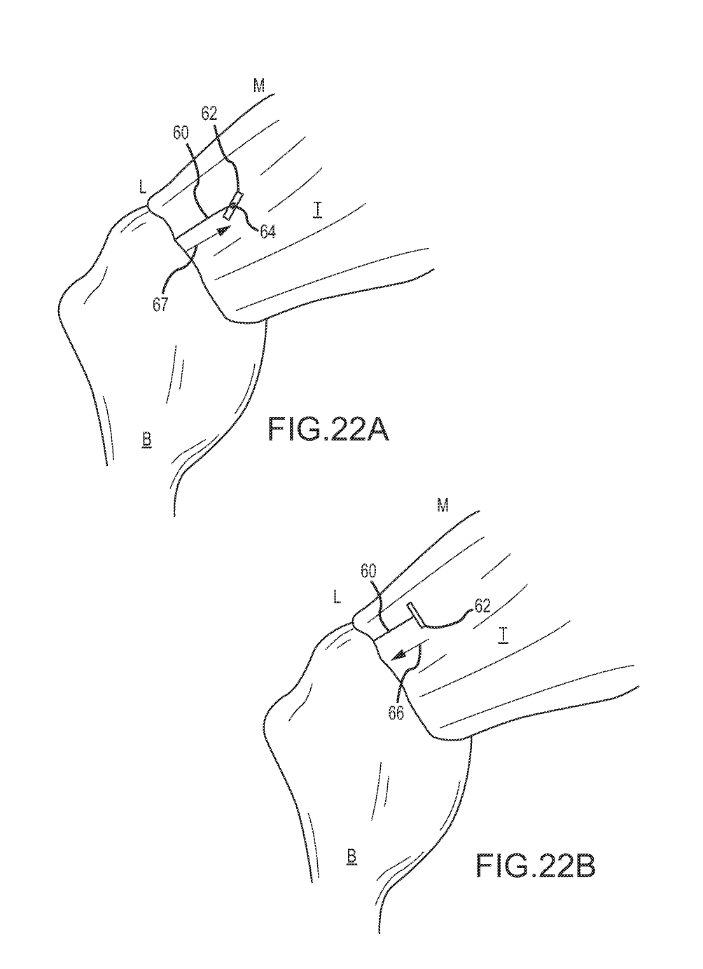

Referring to FIG. 22A, the implant 62 and suture 60 are illustrated in the orientation they are found when the suture is deployed by the needle (not shown). FIG. 22B illustrates the suture 60 and implant 62 after the suture has been deployed, and tension applied in the medial to lateral direction, as illustrated by the directional arrow the 66. When tension is applied, the implant 62 will naturally move to a rotated position so as to embed itself within the surrounding tissue, and thereby providing an anchor point to which the distal end of the suture is attached.

Referring to FIGS. 23A and 23B, the suture 60 is illustrated with a pair of implants 62. More specifically, for FIG. 21A, the suture 60 and implants are shown as they are emplaced by a needle (not shown) in the lateral to medial direction, as indicated by the directional arrow 67. FIG. 23B illustrates the suture 60 and the pair of implants after tension is applied to the suture, thereby resulting in the implants rotating to their embedded orthogonal positions. By placing more than one implant in series along the length of the suture, one can appreciate the flexibility provided to a surgeon in terms of pullout capacity for the suture. Both the implant(s) 62 and the suture 60 remain within the center mass or thickness of the targeted tissue, again maximizing surface contact with the tissue for optimizing the repair capability.

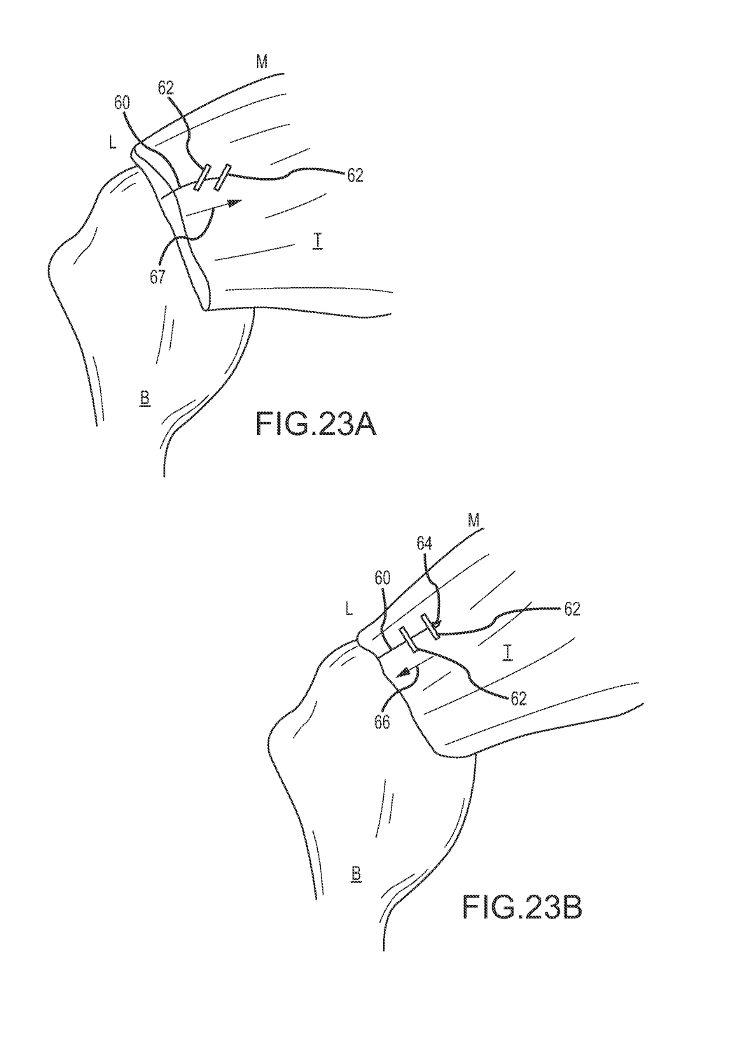

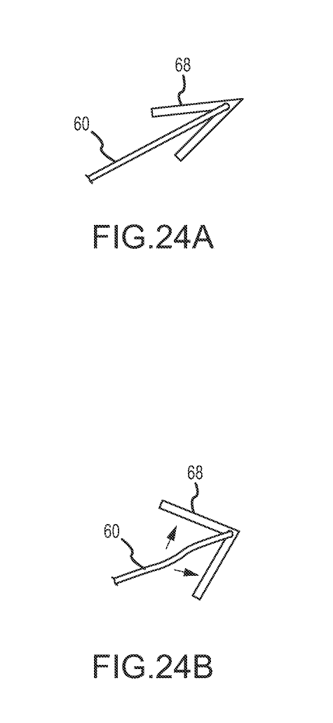

FIGS. 24A and 24B illustrate another type of implant design with a suture, namely, a suture 60 attached to an implant 68, and further in which the implant is in a folded or retracted position as shown in the FIG. 24A, and an expanded or deployed position shown in the FIG. 24B. The implant 68 is intended to be deployed by a cannulated needle (not shown) such that when the implant is in the folded position, it is stored within the needle, and when the implant extends beyond the needle, the implant deploys, and the wings of the implant spread apart.

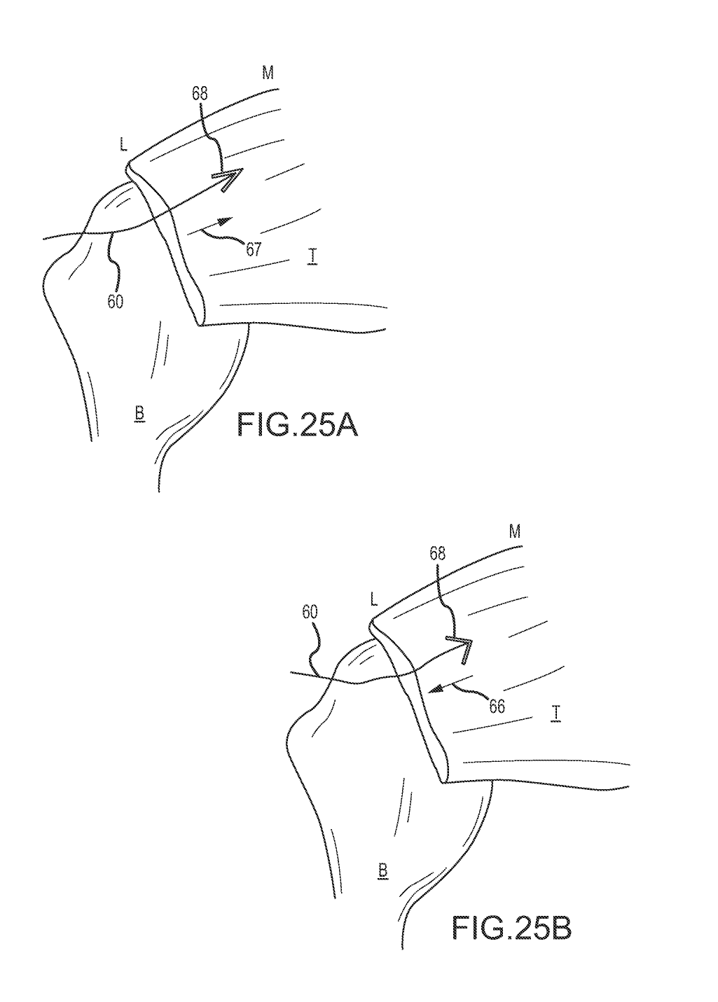

Referring to FIGS. 25A and 25B, these figures show use of the implant 68. More specifically, this FIG. 25A illustrates the position of the implant 68 as it is placed within the rotator cuff tendon (introducer needle not shown). When the introducer needle is removed, the implant 68 moves to the expanded or deployed position and therefore, is anchored within the tissue of the rotator cuff as shown in the FIG. 25B.

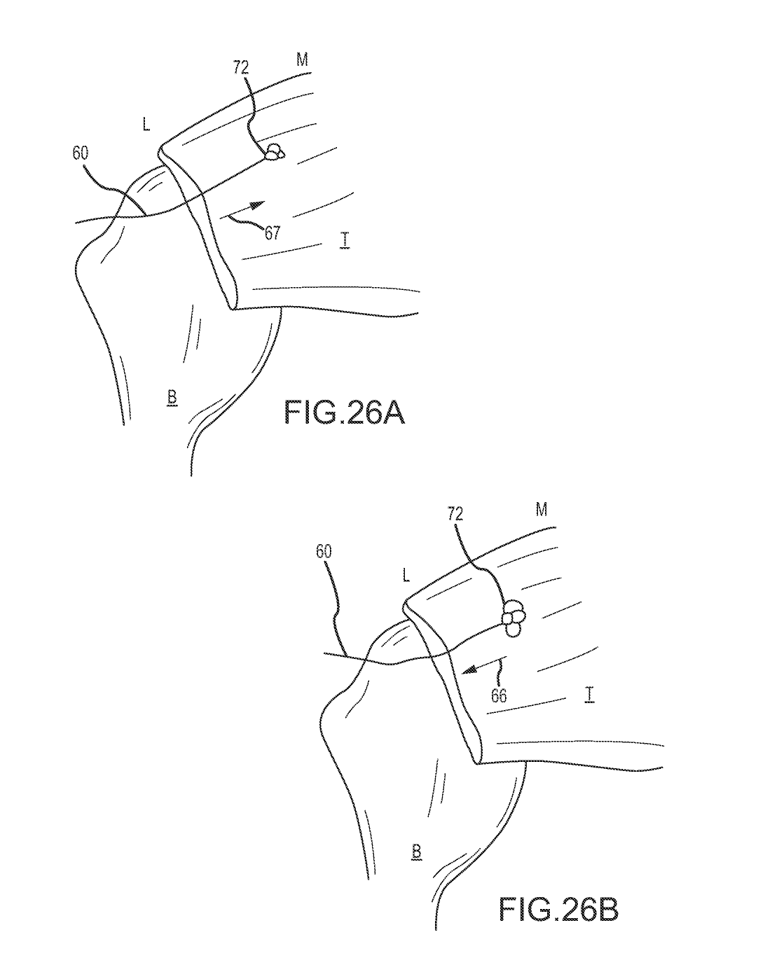

Referring to the FIG. 26A and FIG. 26B, a similar implant 72 is illustrated in which the implant 72 is shown in FIG. 26A in a stored position, such as within an introducer needle (not shown), and the FIG. 26B illustrates the implant 72 when deployed. The FIGS. 26A and 26B are intended to illustrate an implant 72 which includes a plurality of knots such that when the knots are held within the cannula of the needle, the knots are compressed against one another and therefore occupy a relatively small volume. However, when the anchor 72 is deployed, the knots and separate from one another, therefore creating an anchor effect in which the expanded volume of the knots provide a mass of material that is able to be retained within the tissue. One advantage of this knot type implant 72 is that it is well-suited for use in soft tissue in which the anchor itself can be simply made of the same material as the suture 60, the compressed knot arrangement transforming into an effective anchor upon deployment.

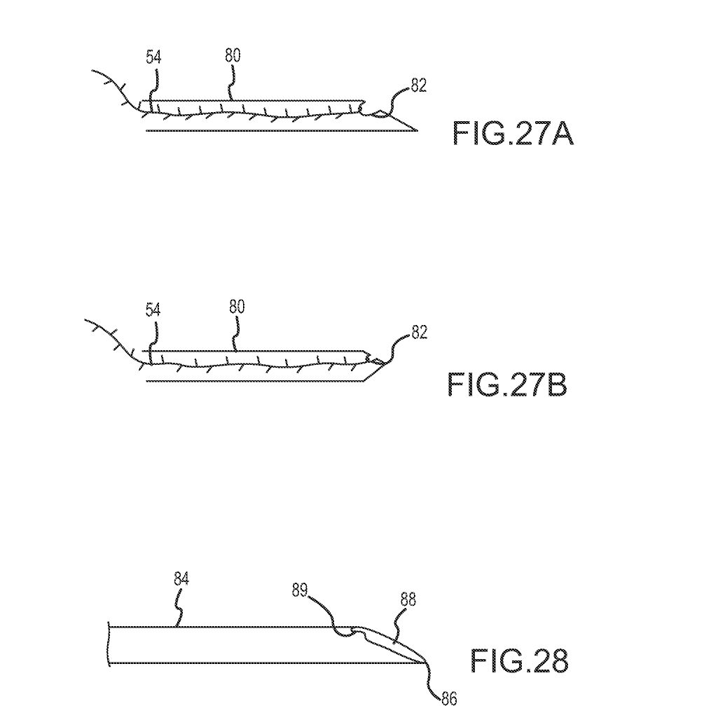

Referring to FIGS. 27A and 27B in yet another embodiment of the invention, two types of suture passing needles 80 are illustrated as alternatives to traditional cannulated introducer needles or suture passing needles. Referring to FIG. 27A, the needles 80 is not cannulated, but rather, has a cut-out or notch 82 that is used to secure a suture 54, and the notch 82 holds the suture as it is advanced into the tissue. As shown, the notch 82 is designed such that it will maintain and hold the suture while the needle is deployed, but the suture will be released when the needle is retrieved or retracted in the direction opposite from advancement. The configuration of the needle 80 in FIG. 27A shows the notch 82 formed on an inclined distal tip. The notch 82 in the needle of FIG. 27B is also formed on an inclined surface at the distal tip; however, the needle at the distal tip incorporates two converging surfaces, similar to a trocar point. In either case, the suture 54 is effectively held in the notch 82 during deployment of the suture. Although a barbed type suture 54 is illustrated, it shall be understood that the needles 80 are well adapted to hold many different type of suture constructions. The needles 80 are also intended to be used with a suture passing device in which the needles are easily loaded within the barrel of the device, and can be deployed like traditional suture passing needles.

FIG. 28 illustrates one example of a modified cannulated needle 84 that can be used for pre-loading of implants and sutures of the present invention. More specifically, the cannulated needle 84 allows for implants and suture to be loaded prior to advancing the needle, and then the implants and sutures are deployed into tissue once the needle is retracted. As shown in the FIG. 28, the needle 84 has a sharp distal tip 86, and the end of the needle is cut at an angle thereby producing an elongated, oblong opening 88 that assists in deployment of the sutures and implants by providing a larger exit area as compared to a round distal opening. FIG. 28 also shows a slot 89 formed in the needle that can be used to hold an anchor during use of the needle, as also explained below with reference to FIG. 31.

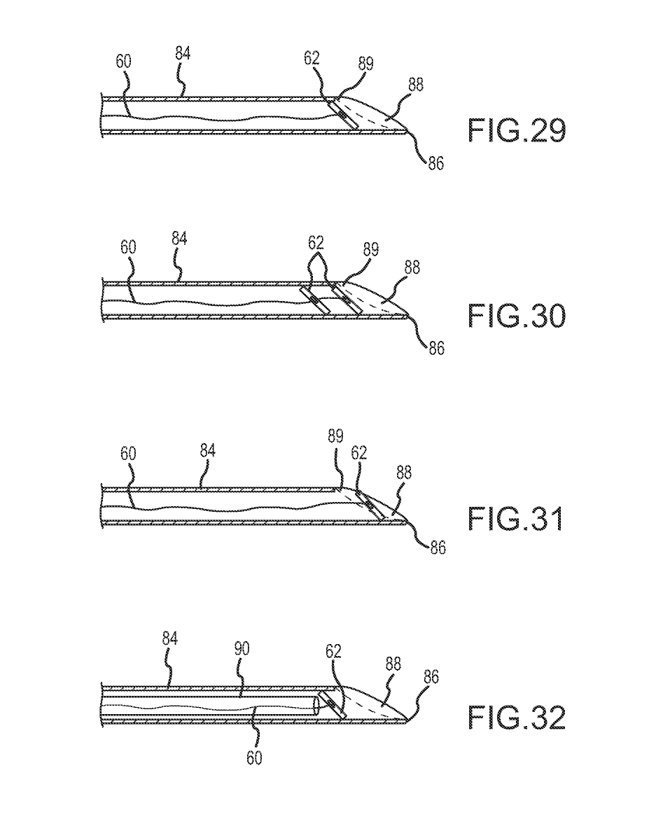

FIG. 29 is a cross-section of the needle shown in FIG. 28 illustrating one example of an implant 62 and suture 60 that can be preloaded within the needle 84. As shown, the implant 62 and suture 60 are easily loaded and held within the needle 84.

Referring to FIG. 30, this cross-section shows the ability of the needle 84 to hold suture 60 and multiple implants 62 arranged in series. One can appreciate that there can be a number of different combination of implants that can be preloaded within the needle, such as additional implants provided in series to selectively provide the desired resistance against pullout of the suture 60.

Referring to the cross-section of FIG. 31, the cannulated needle is illustrated in yet another configuration in which the slot 89 is used to hold the implant 62. When the needle is advanced, the implant 62 remains within the slot 89, but when the needle is retracted, the implant 62 becomes imbedded in the tissue surrounding the tip of the anchor exposed at the slot 89, and therefore the needle separates from the anchor as discussed below. The implant 62 is not fully enclosed within the needle 84 in a loose manner, but rather, is held within the slot 89 with some degree of friction to ensure that the implant 62 can be placed at the desired location within the targeted tissue. However, the frictional engagement of the implant 62 within the slot 89 is not so great that the implant 62 does not easily release from the slot. The implant 62 has an exposed tip extending beyond the diameter of the needle as shown such that when the needle is withdrawn, the exposed edge will automatically embed within the surrounding tissue, thereby providing an anchoring force that easily allows the implant to be removed within the slot. Further, this exposed tip or edge of the implant ensures that the implant is slightly rotated in a counterclockwise direction as the needle is withdrawn, therefore best orienting the implant within the tissue in a substantially perpendicular direction as compared to the axis or extension of the attached suture. Therefore, the implant is optimally deployed within the tissue.

FIG. 32 is another cross-section that illustrates yet another method of deploying suture and an implant. More specifically, the FIG. 32 illustrates the needle 84, in combination with a deployment sleeve 90 that is used to hold the suture and implant at the desired location within the needle. The deployment sleeve 90 is illustrated in which the implant 62 is positioned beyond the distal end of the deployment sleeve 90. The sleeve is therefore contained within the needle while the needle advances into the tissue, and when the needle is fully deployed, the sleeve is used to hold the implant in place. Therefore, the sleeve 90 provides some structure for the surgeon to use in order to exactly place the suture and implant at the precise location, and to ensure that the anchor or suture will not shift as the needle is withdrawn. The sleeve therefore allows the implant and suture to engage the tissue and provide optimum resistance for the repair. Once the implant is set within the tissue, the sleeve can then be withdrawn, therefore leaving only the suture and implant.

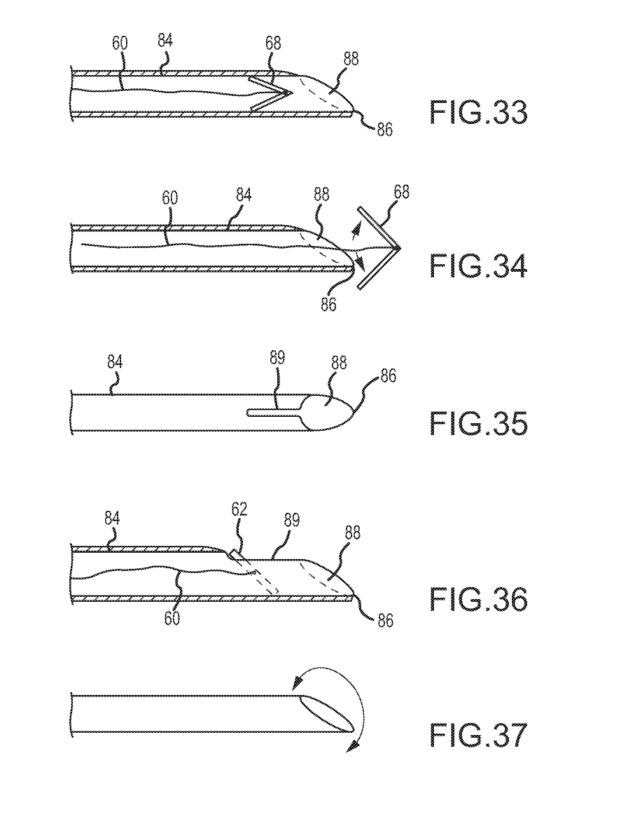

Referring to cross-sections of FIG. 33 and FIG. 34, these figures illustrate the cannulated needle 84 as it can be used to introduce the implant 68. Referring to FIG. 33, it is shown that the implant 68 is constrained within the needle in a folded position. The FIG. 34 illustrates the implant 68 after the needle has been withdrawn, therefore allowing the implant 68 to move to its spread or deployed position thereby serving as an effective anchor within the targeted tissue.

Referring now to the cross-sections of FIG. 35 and FIG. 36, the needle 84 is illustrated with the slot 89 that is used to hold an implant, such as implant 62. FIG. 35 is a plan view showing that the slot 89 may have a selected length and width, and the FIG. 36 is cross-sectional side view showing the implant 62 loaded within the slot 89. The slot 89 may be dimensioned to most optimally frictionally hold a selected implant. Therefore, it should be apparent that the slot 89 allows the implant to be locked into place for employment. In the FIG. 36, the implant has a length such that when loaded, it does not have an appreciable length that extends beyond the diameter of the needle as compared to the slot configuration in the FIG. 31. However, the exposed edge will still automatically embed within the surrounding tissue since the tip of the anchor is exposed and will be in contact with surrounding tissue, thereby providing an anchoring force which easily allows the implant to be removed within the slot 89.

FIG. 37 is intended to represent a feature of the needle 84 that allows it to be loaded into the handle of the device, and subsequently deployed in any desired angular orientation. Specifically, the needle 84 may be somewhat flexible, which enables the needle to be deployed through the barrel of the device, and which the barrel itself may have a slight curve or angular orientation in order to most optimally deploy suture and anchors. Therefore, it is also contemplated that the device of the invention may also include a shaft that is a non-linear, and may have one or more passageways with slight bends or curves especially adapted for orienting needles in optimal angular orientations.

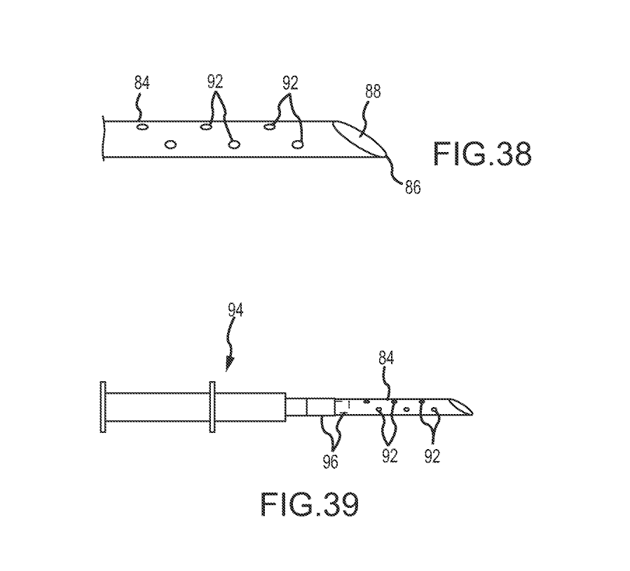

FIG. 38 illustrates another feature of the needle 84 in another embodiment that has a plurality of perforations or fenestrations 92. These perforations 92 can be used to allow for administration of medications or other agents into the targeted soft tissue. For example, often times in a rotator cuff or other similar soft tissue repairs, it is advantageous to be able to inject healing agents to aid in the success of the repair. However, it is often difficult to access the desired tissue with an additional needle. Therefore, the same needle that is used to introduce suture and anchors can also be used to administer the medications/agents. When the needle is deployed into tissue, an injection can be administered simply by attaching a common syringe to the proximal end of the needle as shown in the FIG. 39. Accordingly, FIG. 39 illustrates a connection that can be utilized to interconnect a syringe 94 to the needle 84. More specifically, the FIG. 39 shows that the diameter of the needle 84 may be sized to frictionally engage one or more of the distal flanges 96 on the syringe. Therefore, a simple friction fit can be established such that when the syringe is squeezed, the liquid agent within the body of the syringe is easily passed from the syringe 94 directly into the cannulated passageway of the suture passing needle 84.

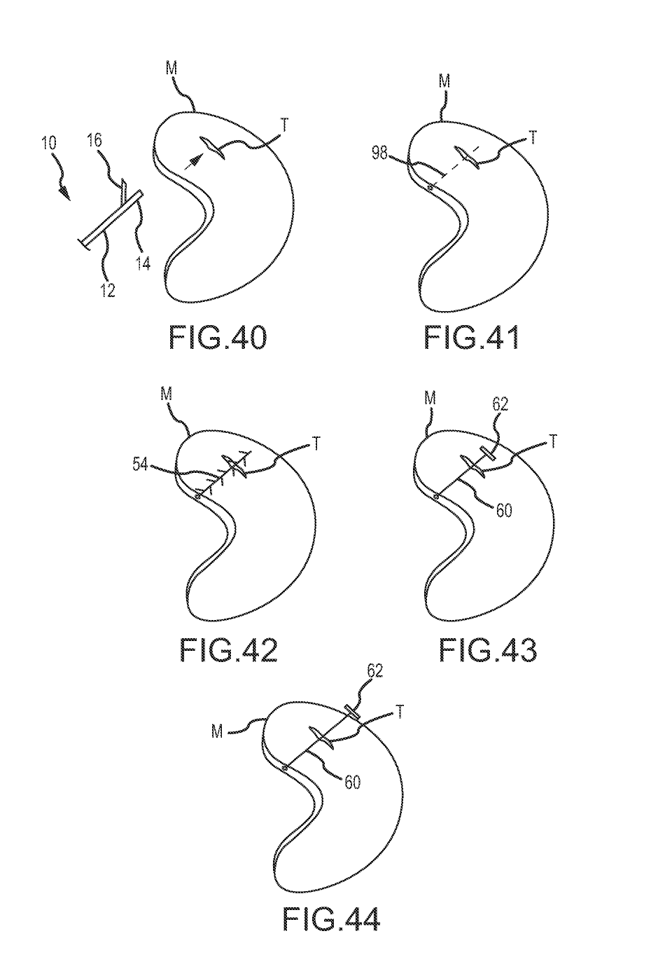

In yet another embodiment of the invention, it is contemplated that the device of the present invention can be used to repair a number of other types of soft tissues, such as a meniscus with a tear. Referring to the FIG. 40, it shows a schematic of the distal end of the suture passing device 10 approaching a meniscus M in the same manner it approaches a rotator cuff, namely, in a direction through the thickness of the meniscus, as opposed to orthogonal or perpendicular to the thickness of the meniscus. More specifically, the FIG. 40 illustrates the meniscus M, and the jaws of the device that are moved toward the tear T in the meniscus M. Referring to FIG. 41, one desired orientation for the needle to place the anchor and suture is shown as the dotted line 98, As shown, the line is oriented substantially perpendicular to the primary direction of the tear T in the meniscus M, and the line extends through the thickness of the meniscus M.

Referring to FIG. 42, this figure shows the meniscus tear T has been repaired with a barb type suture 54. Referring to the FIG. 43, the meniscus M is illustrated in this figure with repair of the tear T by the implant 62, and in which the implant 62 remains within the meniscus tissue. Referring to FIG. 44, the meniscus M is shown as being repaired with the implant 62 in which the implant is deployed outside of the edge of the meniscus M. In FIGS. 42-44, the sutures and implants are shown in solid lines for clarity, but it shall be understood that the sutures and implants would not be visible with the exception of the point of entry for sutures, and the exposed implant in the FIG. 44.

From the foregoing, the present invention provides a number of inventions in combinations and sub-combinations for introducing suture and implants into targeted tissue for repair. The invention comprises not only a suture passing device with many different features, but also various forms of sutures and implants, constructions for suture passing needles, methods of deploying needles with loaded sutures and implants, in which each are described herein as separate embodiments of the invention.

What is claimed is:

1. A suture passing device, comprising:
a pair of handles;
a shaft operably connected to the handles;
a trigger mechanism operably connected to the shaft;
said shaft having a distal end including a stationary jaw and a movable jaw controlled for movement between and open position and a closed position by manipulating said handles;
said shaft having an opening to receive a suture passing needle and attached suture;
said shaft extending along a longitudinal axis; and wherein.
said needle being advanced by operating the trigger mechanism in which the needle is advanced in a direction parallel to the longitudinal axis of the device through the opening in the shaft and into targeted tissue.

2. A method of passing suture into targeted tissue, said method comprising:
providing a suture passing device, comprising:
a pair of handles;
a shaft operably connected to the handles;
a trigger mechanism operably connected to the shaft;
said shaft having a distal end including a stationary jaw and a movable jaw controlled for movement between and open position and a closed position by manipulating said handles;
said shaft having an opening to receive a suture passing needle and attached suture;
said shaft extending along a longitudinal axis; and
advancing the needle through the device by operating the trigger mechanism, wherein the needle is advanced in a direction parallel to the longitudinal axis, and through the opening in the shaft into the targeted tissue.

3. A method of passing suture into targeted tissue for a rotator cuff repair, said method comprising:
providing a suture passing device, comprising:
a pair of handles;
a shaft operably connected to the handles;
a trigger mechanism operably connected to the shaft;
said shaft having a distal end including a stationary jaw and a movable jaw controlled for movement between and open position and a closed position by manipulating said handles;
said shaft having an opening to receive a suture passing needle and attached suture;
said shaft extending along a longitudinal axis; and
advancing the needle through the device by operating the trigger mechanism, wherein the needle is advanced in a direction parallel to the longitudinal axis, and through the opening in the shaft into the targeted tissue of the rotator cuff, near a center of mass of the rotator cuff, and the needle advancing lateral to medial through a thickness of the rotator cuff.

4. A device, as claimed in claim 1, wherein:
said opening of said shaft includes a plurality of openings extending substantially parallel to one another through said shaft, thereby enabling a corresponding plurality of suture passing needles to be passed simultaneously into targeted tissue by operation of said trigger mechanism.

5. In combination, a suture and anchor repair system, especially adapted for repair of torn muscles or tendons, said combination comprising:
at least one suture extending through a thickness of a targeted muscle/tendon, and in a direction substantially parallel to a direction of extension of the targeted muscle/tendon;
at least one anchor secured to a bone structure adjacent the targeted muscle/tendon, and said at least one suture attached to said anchor.

6. A suture construction comprising:
a length of suture, and a plurality of barbs or protrusions extending angularly away from a longitudinal axis of said length of suture, said barbs or protrusions being spaced from one another along a length of said length of suture.

7. A method of deploying an anchor used with a length of suture for preparing targeted tissue, said method comprising:
advancing the suture and anchor in a direction through a suture passing device, wherein the suture and anchor are passed into the tissue in a direction substantially parallel to a longitudinal axis of a shaft of the suture passing device;
placing the suture and anchor at a desired location within the targeted tissue;
placing tension on the suture in a direction opposite to the direction of advancement; and
rotating the anchor so that it is disposed substantially perpendicular to the direction of extension of the suture.

8. A method of deploying an anchor used with a length of suture for preparing targeted tissue, said method comprising:
advancing the suture and anchor through a suture passing device, wherein the suture and anchor are passed into the tissue in a direction substantially parallel to a longitudinal axis of a shaft of the suture passing device;
placing the suture and anchor at a desired location within the targeted tissue;
removing the anchor from within the shaft, wherein the anchor moves from a stored position within the shaft, to a deployed position within the targeted tissue, the deployed position resulting in expanding at least one dimension of the anchor.

9. A suture passing needle, comprising:
a linear extending needle shaft having an opening extending through the shaft;
said shaft having a sharp and distal tip, and a notch formed in the distal tip.

10. A suture passing needle, as claimed in claim 9, further including:
a plurality of perforations extending through said needle shaft

11. In combination, a system for deploying an anchor and suture into targeted tissue, said system comprising:
a shaft of a suture passing device having an opening extending through the shaft;
a deployment sleeve extending through said opening;
a length of suture extending through said sleeve, and having a distal end extending beyond a distal end of said deployment sleeve; and
an anchor attached to the distal end of said suture.
